(19) United States
(12) Patent Application Publication
(10) Pub. No.: US 2017/0304430 A1
(43) Pub. Date: Oct. 26, 2017

(54) VIRUS VECTOR, CELL, AND CONSTRUCT

(71) Applicant: Kyushu University, National University Corporation, Fukuoka-shi, Fukuoka (JP)

(72) Inventors: Kenzaburo TANI, Fukuoka-shi, Fukuoka (JP); Takafumi HIRAMOTO, Fukuoka-shi, Fukuoka (JP); Makoto TAKEDA, Tokyo (JP); Maino TAHARA, Tokyo (JP)

(21) Appl. No.: 15/523,547

(22) PCT Filed: Nov. 5, 2015
(86) PCT No.: PCT/JP2015/081145

§ 371 Date: May 1, 2017

(30) Foreign Application Priority Data
Nov. 5, 2014 (JP) 2014-225642

(51) Int. Cl. A61K 39/165; A61L 27/38; A61K 48/00; C12N 15/86; C12N 7/00; C07K 14/005; A61K 39/00

(52) U.S. Cl. CPC A61K 39/165; C12N 7/00; A61L 27/3834; C07K 14/005; A61K 48/0058; C12N 15/86; A61K 2039/5158; C12N 2760/18422; C12N 2760/18434; C12N 2760/18443

(57) ABSTRACT

A virus vector contains a genome that is derived from a virus belonging to the family Paramyxoviridae, and has a gene encoding a modified H protein, a gene encoding a modified F protein, and foreign genes. The genome may be segmented into multiple segments, and each genome segment may have a leader sequence and a trailer sequence.

### TECHNICAL FIELD

The present disclosure relates to a virus vector, a cell, and a construct.

### BACKGROUND ART

Because of low invasiveness upon collection and administration, blood cells are most frequently applied clinically as therapeutic cells for regenerative medicine or the like. Particularly hematopoietic stem cells and hematopoietic progenitor cells have been used for treatments of hematopoietic malignancies over the past about 30 years, and are currently used for general treatments. Clinical trials have been conducted using, in addition to these cells, blood cells including lymphocytes, NK cells, NKT cells, dendritic cells, and the like, as cells for immunocellular therapy against malignant cells.

Furthermore in recent years, with regard to gene therapy using gene-modified blood cells, clinical trials have been conducted concerning severe congenital diseases or malignancies. In particular, treatments using gene-modified T cells transduced with a T cell receptor or chimeric antigen receptor gene have received attention as novel treatments with potentially significant therapeutic effects against malignancy.

Currently, a lentivirus vector or a retrovirus vector is used for transduction into T cells. However, such a lentivirus vector and a retrovirus vector are problematic in genomic toxicity due to insertion into genomic DNA.

Conversely, methods known as transduction methods that unlikely cause insertion to genomic DNA include methods using adenovirus vectors, as well as methods using non-viral vectors such as mRNA and plasmids. However, adenovirus vectors and non-viral vectors are problematic in extremely low transduction efficiency and limited cell types that can be used.

The use of a Sendai virus vector is also known as a transduction method that unlikely causes insertion into genomic DNA. For example, Patent Literature 1 discloses a Sendai virus vector modified through deletion of an F gene, so as to avoid the release of secondary infectious viral particles. Furthermore, Patent Literature 2 discloses a Sendai virus vector carrying a gene involved in cell reprogramming.

Measles virus belonging to the family Paramyxoviridae, to which Sendai virus also belongs, is a virus retaining extremely high infectivity to immunocytes and epithelial cells. Patent Literature 3 discloses measles virus retaining multiple genome segments, at least one of which contains a foreign gene that can be expressed within a host.

### CITATION LIST

### Patent Literature

Patent Literature 1: Japanese Patent No. 3602058

Patent Literature 2: International Publication No. WO 2010/008054

Patent Literature 3: International Publication No. WO 2007/007921

### SUMMARY OF INVENTION

### Technical Problem

When a gene is introduced into a T cell, the Sendai virus vectors disclosed in Patent Literature 1 and Patent Literature 2 above are inconvenient in that the construction of the Sendai virus vectors is relatively difficult, and Sendai virus tends to remain and the removal thereof is difficult. Moreover, there is less knowledge about how the Sendai virus vectors affect humans that are not original hosts for Sendai virus. Actual clinical use of the Sendai virus vectors should be carefully performed because of safety concerns.

Furthermore, similarly to adenovirus vectors, in the case of the Sendai virus vectors disclosed in the above Patent Literature 1 and Patent Literature 2 and the measles virus disclosed in the above Patent Literature 3, T cells should be stimulated and activated prior to transduction in order to increase transduction efficiency. This causes a concern of inducing T cell differentiation due to T cell activation, and thus transduction into naive T cells, undifferentiated B cells, or the like is difficult.

As described above, conventional virus vectors are problematic in that the safe use of these vectors for humans is insufficiently ensured and cells into which genes can be efficiently introduced by the vectors are limited.

The present disclosure has been achieved in view of the above circumstances and an objective thereof is to provide a virus vector, a cell, and a construct that are highly safe and enables efficient introduction of genes into wide-ranging cells.

### Solution to Problem

A virus vector according to a first aspect of the present disclosure includes

a genome derived from a virus belonging to the family Paramyxoviridae in which both a gene encoding an H protein and a gene encoding an F protein are modified, wherein

the genome contains a foreign gene.

In this case, the genome

may be segmented into multiple segments, wherein

each genome segment may include a leader sequence and a trailer sequence.

Furthermore, the genome may be segmented into two segments.

The modifications may be

a deficiency in the gene encoding the H protein, or a mutation resulting from substitution, deletion, or addition of one or several amino acids of the H protein, and

a deficiency in the gene encoding the F protein, or a mutation resulting from substitution, deletion, or addition of one or several amino acids of the F protein.

Furthermore, a gene encoding an M protein in the genome may include,

a mutation resulting from substitution, deletion, or addition of one or several amino acids of the M protein.

Furthermore, the virus belonging to the family Paramyxoviridae may be measles virus.

Furthermore, the foreign gene may include

OCT3/4, SOX2 and KLF4.

A cell according to a second aspect of the present disclosure is a cell, wherein

the foreign gene is introduced by the virus vector according to the first aspect of the present disclosure.

In this case, the cell according to the second aspect of the present disclosure may be a blood cell including a hematopoietic stem cell.

In addition, the blood cell may be

a naive T cell, a stem cell-like memory T cell, or an undifferentiated B cell.

Furthermore the foreign gene may include,

OCT3/4, SOX2 and KLF4, and

the cell may be a pluripotent stem cell in a ground state.

A construct according to a third aspect of the present disclosure, contains

a nucleic acid that is a template for multiple genome segments derived from a virus belonging to the family Paramyxoviridae, in which a leader sequence is located at the 3′ end and a trailer sequence is located at the 5′ end, respectively, wherein

both the gene encoding the H protein and the gene encoding the F protein in the segmented genome are modified.

### Advantageous Effects of Invention

According to the present disclosure, a gene can be efficiently introduced into wide-ranging cells with a high level of safety.

### BRIEF DESCRIPTION OF DRAWINGS

FIG. 1 shows the composition of a segmented genome of a non-transmissible measles virus vector carrying six genes;

FIG. 2 shows the composition of the measles virus vector plasmid shown in FIG. 1;

FIG. 3 shows fluorescent images of BJ cells and activated T cells into which measles virus vectors were introduced;

FIG. 4 shows the results of analyzing the expression of genes carried by the measles virus vector;

FIG. 5 shows the transduction efficiencies of the measles virus vector and a Sendai virus vector into cells of each blood cell lineage;

FIG. 6 shows the transduction efficiencies of the measles virus vector and the Sendai virus vector into cord blood-derived T cells;

FIG. 7 shows the transduction efficiencies of the measles virus vector and the Sendai virus vector into each fraction of peripheral blood-derived T cells;

FIG. 8 shows the phase difference and fluorescent images of the colonies of induced pluripotent stem cells (iPS cells) established from BJ cells;

FIG. 9 shows the phase difference and fluorescent images of the primary colonies of iPS cells established from T cells;

FIG. 10 shows the expression of undifferentiation markers in BJ cell-derived iPS cells;

FIG. 11 shows the results of analyzing the expression of undifferentiation markers and virus-derived genes by an RT-PCR method;

FIG. 12 shows the results of analyzing the in vitro induction of triploblastic differentiation;

FIG. 13 shows the results of analyzing triploblastic differentiation ability by a teratoma formation test;

FIG. 14 shows the results of analyzing the karyotype of BJ cell-derived iPS cells; and

FIG. 15 shows the morphology of iPS cells in a ground state.

### DESCRIPTION OF EMBODIMENTS

Embodiments according to the present disclosure will be explained as follows.

### Embodiment 1

First, Embodiment 1 is explained in detail. A virus vector according to this embodiment contains a genome derived from a virus belonging to the family Paramyxoviridae.

The virus belonging to the family Paramyxoviridae (hereinafter, also simply referred to as “virus”) is an RNA virus having a (−) strand RNA genome. Examples of the virus include Measles virus, Sendai virus, Newcastle disease virus, Mumps virus, Parainfluenza virus 1, Parainfluenza virus 2, Parainfluenza virus 3, Parainfluenza virus 5 or Simian virus 5, Metapneumo virus, Respiratorysyncytial virus, Rinderpest virus, and Distemper virus. The virus belonging to the family Paramyxoviridae is preferably measles virus.

The genome of the virus belonging to the family Paramyxoviridae is unsegmented single-stranded RNA, having a leader sequence (Le) at the 3′ end and a trailer sequence (Tr) at the 5′ end. Various genes encoding functional proteins that constitute the virus are located between the leader sequence and the trailer sequence. The leader sequence has promoter activity. The trailer sequence has promoter activity in the case of antigenome.

Major genes encoding the functional proteins are a gene encoding an N protein (N gene), a gene encoding a P protein (P gene), a gene encoding an M protein (M gene), a gene encoding an F protein (F gene), a gene encoding an H protein (H gene), and a gene encoding an L protein (L gene). Transcriptional regulatory sequences; that is, a transcription initiation sequence and a transcription termination sequence, are present on both ends of each of the six genes in the genome.

The N proteins are systematically aligned and bound in order from the 5′ end with respect to the viral genome, so as to package the genome RNA. The P protein functions as a small subunit of RNA-dependent RNA polymerase, and is involved in transcriptional replication of the viral genome. In addition, accessory proteins such as a V protein and a C protein may be synthesized from the P gene through RNA editing mechanism or the like. The L protein functions as a large subunit of RNA-dependent RNA polymerase, and is involved with the P protein in transcriptional replication of the viral genome.

The M protein is a matrix protein supporting from inside the structure of viral particles. The F protein is involved in cell fusion, and imparts pathogenicity to a virus. The H protein is a viral receptor-binding protein. The H protein binds to signaling lymphocyte activation module (SLAM) or Nectin4 as a receptor for a wild-type virus to infect. SLAM and Nectin4 are expressed only in limited cell types, thereby limiting viral host range.

The term “genome derived from a virus belonging to the family Paramyxoviridae” means a genome that has genes encoding the functional proteins, and can be amplified by infected cells.

In the above genome contained in the virus vector according to this embodiment, both the H gene and the F protein are modified. Modification of the H gene is a deficiency in the H gene, for example. Deletion of the H gene can cause the absence of the expression of the H protein. Moreover, modification of the H gene may be a mutation resulting from substitution, deletion, or addition of one or several amino acids of the H protein.

More specifically, examples of the modification of the H gene include, substitution of the 390^{th} asparagine with isoleucine (N390I), substitution of the 416^{th} asparagine with aspartic acid (N416D), substitution of the 446^{th} threonine with serine (T446S), substitution of the 481^{st} asparagine with tyrosine (N481Y) and substitution of the 492^{nd} glutamic acid with glycine (E492G) of the H protein. Because of these substitutions, the H protein can bind to a membrane co-factor protein (CD46), the expression of which is observed in almost all cells, in addition to SLAM and Nectin4.

The structure or the functions of the H protein can be changed with respect to the wild-type H protein through the modification of the H gene. Since the H protein is a viral receptor-binding protein as described above, the virus vector can use molecules other than SLAM and Nectin4 as receptors through modification of the H gene. Specifically, modification of the H gene can result in increased number of cell types that the virus vector can infect.

Modification of the F gene is deficiency in the F gene, for example. Deletion of the F gene can cause the absence of the expression of the F protein. For example, when the genome of measles virus is used, the F protein-deficient virus vector is unable to produce any pathogenic virus within cells other than special cell lines (Vero/SLAM/F cell). Hence, unless caused to infect the above special cell lines, the virus vector can be caused to lose the transmission.

Modification of the F gene may be a mutation resulting from substitution, deletion, or addition of one or several amino acids of the F protein. The structure or the functions of the F protein may be changed with respect to the wild-type F protein through the modification.

Furthermore, the gene encoding the M protein in the above genome may have a mutation resulting from substitution, deletion, or addition of one or several amino acids of the M protein. Examples of the mutation include substitution of the 64^{th} proline with serine (P64S) and substitution of the 89^{th} glutamic acid with glycine (E89G) of the M protein. Since the M protein is a matrix protein supporting from inside the structure of viral particles, the ability to form viral particles can be improved and the cell fusion ability can be lowered by mutating the M protein.

The above genome contained in the virus vector according to this embodiment has foreign genes. Therefore, the virus vector enables the expression of the foreign genes. Examples of the foreign genes include, but are not limited to, genes encoding various proteins of viruses, bacteria, parasites and the like, which induce pathogenicity, genes encoding various cytokines, genes encoding various peptide hormones, and genes encoding cell reprogramming factors. In addition, as a foreign gene, a reporter gene such as a gene encoding a Green Fluorescent Protein (GFP) or an Enhanced Green Fluorescent Protein (EGFP) may be inserted.

The number of foreign genes is not particularly limited, and preferably ranges from 2 to 6. Specific examples of foreign genes include reprogramming factors such as OCT3/4, SOX2 and KLF4. Other reprogramming factors such as L-MYC and PIN1 may of course be further inserted as foreign genes into a genome.

The positions of genes encoding the above functional proteins and foreign genes in a genome are not particularly limited, as long as the positions are between a leader sequence and a trailer sequence. The mutual positional relationship among genes encoding the above functional proteins in a genome can be arbitrarily determined regardless of positions such as the order of alignment in a wild-type viral genome.

The above genome contained in the virus vector according to this embodiment may be segmented into multiple segments. The term “segmentation” means that genome RNA is fragmented into multiple RNA fragments. Specifically, multiple genome segments form a set of RNA groups that function as one genome. The number of genome segments is not limited, but is preferably up to 6, and is particularly preferably 2.

In this case, each genome segment has a leader sequence and a trailer sequence. Preferably, a leader sequence is located at one end and a trailer sequence is located at the other end of each genome segment. In this case, any one of genes encoding the above functional proteins, and foreign genes are placed between the leader sequence and the trailer sequence of each genome segment.

Genes encoding functional proteins and foreign genes may be inserted into one, two or more of multiple genome segments. When multiple foreign genes are expressed, all the multiple foreign genes may be inserted into one of multiple genome segments. Alternatively, one or more foreign genes may be inserted into each of two or more of multiple genome segments, so as to enable the insertion of multiple foreign genes overall. When multiple foreign genes are inserted into one of multiple genome segments, the number of foreign genes is not limited as long as the number is within the range that does not cause any significant decrease in the expression efficiency of genes encoding the above functional proteins.

The positions of foreign genes in multiple genome segments are not particularly limited. For example, when the genome of measles virus is segmented into two segments to cause deficiency in the F gene, and reprogramming factors are inserted as foreign genes, the resulting first genome segment contains a leader sequence, OCT3/4, the N gene, the P gene, a modified M gene, SOX2, and a trailer sequence that are placed in this order from the 3′ end to the 5′ end, and the resulting second genome segment contains a leader sequence, KLF4, a modified H gene, the L gene, and a trailer sequence are placed in this order from the 3′ end to the 5′ end. When L-MYC and PIN1 are further inserted as foreign genes, preferably, L-MYC may be inserted between SOX2 and a trailer sequence, and PIN1 may be inserted between the H gene and the L gene.

Genes encoding the above functional proteins, excluding the H gene, the F gene, and the M gene, may be mutated genes, each comprising a nucleotide sequence that is not completely the same as that of the corresponding wild-type gene contained in the genome of the wild-type virus, as long as the activity upon transcription and replication is equivalent to or higher than the wild-type activity. The nucleotide sequences of genes encoding functional proteins may be the nucleotide sequences encoding proteins, each comprising amino acids resulting from deletion, substitution, or addition of one or several (such as 1 to 15, preferably 1 to 8, and more preferably 1 to 5) amino acids, as long as the activity upon transcription and replication is equivalent to or higher than that of each natural protein. The mutant proteins and the natural proteins have amino acid sequence homology of preferably 90% to 100%, for example, and the amino acid homology may range from 40% to 90%, for example, as long as the activity upon transcription and replication is retained.

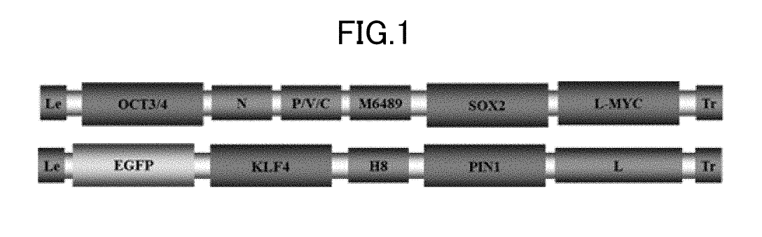

FIG. 1 shows a preferred example of the above segmented genome having genes encoding reprogramming factors and a gene encoding EGFP as foreign genes. In the genome segmented into two segments, the H gene is modified (H8) so as to add N390I, N416D, T446S, N481Y and E492G mutations to the H protein, and the F gene is deficient. Furthermore, the M gene is modified (M6489) so as to add P64S and E89G mutations to the M protein. In addition, the P gene is represented by PN/C.

The virus vector has ability to infect cells and transmissibility. The expression “ability to infect cells” refers to ability to retain adherence of the virus vector to host cells and membrane fusion ability, so as to be able to introduce the viral genome or the like into cells. Furthermore, the term “transmissibility” refers to ability to replicate the genome introduced into cells, and to form infectious particles or complexes similar thereto, so as to be able to transmit the genome to other cells.

The virus vector according to this embodiment can be constructed by a known method for constructing a virus vector. For example, the virus vector can be constructed by rearranging viral particles using cDNA corresponding to a viral genome, or using a viral genome. Here, the expression “rearranging viral particles” means to artificially prepare a viral genome, so as to prepare the original virus or a recombinant virus in vitro or within cells.

Any of these methods generally involves isolating the genome from a virus, and then preparing cDNA of the genome via reverse transcription reaction or the like. When the genome is subsequently segmented, the cDNA is fragmented into multiple cDNA fragments by a known genetic recombination technique, a nucleic acid amplification method and the like, and then the resultant is subjected to a procedure such as insertion of a foreign gene. A method for fragmentation is not limited, as long as multiple cDNA fragments are thus prepared by the method based on the nucleotide sequence or the like of the cDNA prepared from the genome.

An example of a preparation method based on the nucleotide sequence of cDNA prepared from a genome is a method that involves obtaining an amplification fragment by a PCR method or the like using as a template a predetermined region in cDNA prepared from a genome. The predetermined region can be set as appropriate in view of the structure of a genome to be expressed, and is not limited. The predetermined region is preferably set, so that gene fragments encoding viral functional proteins can be separately amplified. When the region is set as described above, the desired types and number of the thus obtained amplified fragments of each gene are bound in order (at positions) as desired, so that cDNA fragments can be prepared.

A foreign gene may be inserted into a segmented genome by inserting a DNA fragment containing a separately prepared foreign gene into the above segmented cDNA using a known genetic recombination technique. A cDNA nucleotide sequence corresponding to the first genome segment containing the N gene and a cDNA nucleotide sequence corresponding to the second genome segment containing the H gene in FIG. 1 are shown in SEQ ID NO: 1 and SEQ ID NO: 2, respectively.

Viral particles may be rearranged using DNA containing a prepared cDNA fragment, preferably plasmid DNA, or, RNA obtained by prior in vitro transcription of cDNA, for example.

In general, direct introduction of the genome of a virus belonging to the family Paramyxoviridae or the antigenome thereof in the form of naked RNA into host cells does not lead to obtainment of a template for RNA-dependent RNA polymerase. Obtainment of a template requires the presence of the N protein, the P protein and the L protein at the initial stage of an RNA synthetic reaction conducted by the RNA-dependent RNA polymerase, and the formation of a complex (RNP complex) of these proteins and genome RNA. Accordingly, for rearrangement of a virus vector, the N protein, the P protein and the L protein are desirably expressed together, or, a host capable of expressing the N protein, the P protein and the L protein is desirably used. In addition to this, when the F gene is deleted in RNA contained in the above virus vector, a host capable of expressing also the F protein is preferably used.

The above virus vector can be constructed by, for example, introducing the above genome or the cRNA of the genome into a host expressing viral N protein, P protein and L protein. Furthermore, DNA containing the above genome or cDNA that serves as a template for the cRNA of the genome and a transcription unit of the DNA may be introduced into a host expressing viral N protein, P protein and L protein. Moreover, DNA containing the above genome or cDNA that serves as a template for the cRNA of the genome, DNA containing viral N gene, DNA containing viral P gene, DNA containing viral L gene, and transcription units of these DNAs may be introduced into a host.

Proteins to be used as the above N protein, P protein and L protein that are expressed by a host may be proteins that are not completely the same as the natural proteins, as long as the proteins have activity equivalent to or higher than that of the natural proteins. For example, the N, P and L proteins may be proteins comprising amino acids resulting from deletion, substitution or addition of 1 or several (such as 1 to 15, preferably 1 to 8, and more preferably 1 to 5) amino acids, or, completely different proteins derived from other viruses or comprising significantly different amino acid sequences.

The above host is not particularly limited, as long as it is a cell capable of expressing the above functional proteins and foreign genes. Examples thereof include cultured mammalian or avian cells, and a chicken egg. Specific examples of cultured cells that can be used herein include BHK-T7/9 cells, CHO, 293 cells, B95a, monkey cells such as COS-7 and Vero, mouse L cells, rat GH3, human FL cells, LLCMK2, MDCK, MDBK, CV-1, HeLa, HepG2, P19, F9, PC1Z, BAF3, Jurkat, human PBMCN, MT-4, Molt-4, NIH3T3, L929, Vero/hSLAM, CHO/hSLAM, A549/hSLAM, HeLa/hSLAM, 293T, BHK, and chicken embryonic fibroblasts. Furthermore, insect cells such as Sf9 cells, and Sf21 cells can also be used.

As the above transcription unit, for example, recombinant vaccinia virus expressing predetermined DNA-dependent RNA polymerase, DNA containing a predetermined DNA-dependent RNA polymerase gene, and the like are preferred.

Furthermore, a virus vector that is obtained by the above manufacturing methods can be replicated selectively and efficiently by co-culture with other cells. For example, in the case of a virus vector constructed using measles virus, cultured cells containing a rearranged virus vector obtained by the above manufacturing methods are seeded on Vero/SLAM/F cells cultured in advance, and then co-cultured, so that giant cells of Vero/SLAM/F cells can be obtained as a result of infection and proliferation of the virus vector.

More specifically, for example, when a measles virus vector is constructed, two plasmids corresponding to genome segments shown in FIG. 1 may be introduced together with another plasmid containing genes encoding functional proteins if necessary, into appropriate cells. Subsequently, cells are recovered and seeded on Vero/SLAM/F cells, and then the thus expressed giant cells are collected, so that a measles virus vector can be obtained. The measles virus vector is caused to infect fresh Vero/SLAM/F cells for amplification, and then the measles virus vector may be released. In such a case, a supernatant containing the relevant virus can be recovered as a viral solution by centrifugation.

As described in detail above, the virus vector according to this embodiment contains a modified H gene, so that the virus vector can infect various cell types, and specifically can broaden the host range. Furthermore, the virus vector contains a modified F gene, so as to be able to prevent the production of the virus having ability to infect cells, and to cause the virus to lose the transmission of the virus vector and thus to improve safety. Moreover, the above virus vector carries out all the replication processes within cytoplasm without DNA synthesis, so that the virus vector has no concern of genomic toxicity, and thus has an extremely high level of safety.

In addition, a genome to be contained in the virus vector according to this embodiment may be segmented into multiple segments. In this manner, the virus vector can carry multiple foreign genes or a gene having a large size. Moreover, each of the genome segments may have a leader sequence and a trailer sequence. Viruses belonging to the family Paramyxoviridae are known to have a characteristic gene expression pattern such that the more downstream (5′ side) location of a gene, the lower the expression of the gene. Each of genome segments has a leader sequence and a trailer sequence, so that polymerase can act on each genome segment, and the expression level of each gene can be increased. Furthermore, multiple foreign genes can be expressed simultaneously in a host. Moreover, segmentation of a genome into two segments leads to a genome size appropriate for maintaining the protein expression efficiency at a high level. An unsegmented single genome can of course exert similar functions.

In addition, in this embodiment, a gene encoding the M protein on a segmented genome may comprise a mutation resulting from substitution, deletion, or addition of one or several amino acids of the M protein. In this manner, the transduction efficiency and the safety of the virus vector for cells can be even more enhanced.

Furthermore, in this embodiment, a virus may be measles virus. Measles virus has strong tropism to immunocytes. Hence, the above virus vector enables highly efficient transduction into B cells, T cells and granulocytes. Furthermore, the above virus vector enables, as described in Examples below, extremely highly efficient transduction into inactivated naive T cells, central memory T cells, effector memory T cells and B cells, and even blood cells including hematopoietic stem cells. Moreover, the original host of the measles virus is a human, and knowledges concerning the effects of the measles virus on humans have been accumulated, and thus the measles virus can be appropriately used with due attention on safety aspects.

In addition, in this embodiment, examples of foreign genes include OCT3/4, SOX2 and KLF4. When the genome of measles virus is used as a genome of the virus vector according to this embodiment, iPS cells in a ground state, the preparation of which has been extremely difficult by a conventional method, can be prepared using the virus vector carrying genes encoding reprogramming factors.

In addition, RNA to be contained in the virus vector according to this embodiment may be, in addition to (−) strand RNA, (+) strand RNA as necessary, similarly to the original genome of the virus.

In another embodiment, a construct appropriate for preparation of a genome to be contained in the above virus vector is provided. The construct contains nucleic acids serving as templates for multiple segments of a genome derived from a virus belonging to the family Paramyxoviridae, in which a leader sequence is located at the 3′ end and a trailer sequence is located at the 5′ end, respectively, wherein both the gene encoding the H protein and the gene encoding the F protein are modified in the segmented genome.

The above nucleic acids may be cDNAs or cRNAs, which are prepared based on each of the above genome segments. For example, the nucleic acids may be multiple cDNAs or plasmid DNAs corresponding to each of the genome segments.

For example, when the genome of measles virus is segmented into two segments to cause deficiency in the F gene, the construct contains cDNA corresponding to a genome in which a leader sequence, the N gene, the P gene, the M gene and a trailer sequence are located in order from the 3′ end to the 5′ end, and cDNA corresponding to a genome in which a leader sequence, the H gene, the L gene and a trailer sequence are located in order from the 3′ end to the 5′ end.

This construct facilitates insertion of foreign genes using a genetic recombination technique of conventional genetic engineering. With the use of the construct for rearrangement of viral particles as described above, the above virus vector containing each genome segment can be efficiently constructed.

This construct can be introduced into prokaryotic cells or eukaryotic cells by a conventional transformation or transfection technique. For example, this expression construct can be introduced into various types of cells, in the form of plasmids.

In addition, nucleic acids to be contained in the above construct may be cRNAs of multiple segments of a genome derived from a virus belonging to the family Paramyxoviridae, in which a leader sequence is located at the 3′ end and a trailer sequence is located at the 5′ end, respectively. Moreover, the above construct contains nucleic acids serving as templates for the genome derived from a virus belonging to the family Paramyxoviridae, in which a leader sequence is located at the 3′ end and a trailer sequence is located at the 5′ end, wherein both the gene encoding the H protein and the gene encoding the F protein in the genome may be modified.

### Embodiment 2

Next, Embodiment 2 is explained. Cells according to this embodiment are transduced with foreign genes by the virus vector according to the above Embodiment 1.

The virus vector can be caused to infect cells by a known method. For example, the virus vector is added to a culture solution containing cells, and then the solution may be centrifuged at room temperature at 1,200×g for 45 minutes. Further examples of a method for causing the virus vector to infect cells include electroporation, lipofection, a heat shock method, a PEG method, a calcium phosphate method and a DEAE dextran method, which involve causing various types of virus to infect cells.

Examples of cells are not particularly limited and include human somatic cells, fibroblasts, and blood cells, as well as monkey somatic cells. Preferred examples of cells include B cells, activated or inactivated T cells, granulocytes, and blood cells including hematopoietic stem cells. Examples of particularly preferred cells include naive T cells, stem cell-like memory T cells or undifferentiated B cells. The titer of the virus vector upon transduction of cells with foreign genes is not particularly limited, and multiplicity of infection (MOI) ranges from 1 to 100, preferably 3 to 20, and more preferably 5 to 10.

Cells transduced with foreign genes by the above virus vector containing at least OCT3/4, SOX2 and KLF4 as the foreign genes express an undifferentiation marker, and are induced to be iPS cells having triploblastic differentiation ability. iPS cells in a ground state can also be induced through transduction by the above virus vector.

As explained in detail above, cells according to the embodiment can express desired multiple foreign genes simultaneously for a long time period, since the genes are introduced by the above virus vector. Moreover, naive T cells, stem cell-like memory T cells or undifferentiated B cells, and even hematopoietic stem cells can be selected as subjects of transduction, so that cells according to this embodiment is extremely useful for gene therapy using gene-modified blood cells, and particularly gene-modified T cell therapy.

Moreover, the cells are transduced with foreign genes by the above virus vector containing OCT3/4, SOX2 and KLF4 and then induced to be iPS cells in a ground state. Such iPS cells in a ground state have even higher proliferation efficiency and are capable of proliferating even when seeded in their unicellular form, so that the iPS cells can be prepared in large amounts and easily stored. Moreover, because of the ground state, the iPS cells can be differentiated into wide-ranging cell types.

In addition, applications of the cells according to this embodiment are varied depending on foreign genes. The cells can be used for T cell therapy against malignancy through the use of a T cell receptor gene or a chimeric antigen receptor gene as a foreign gene to be introduced into the cells. Furthermore, Zinc finger nuclease or the like is introduced as a foreign gene into T cells so as to disrupt (gene editing) a CCRS gene, and thus the cells can be used for treatment of HIV/AIDS. Enzyme deficiency can also be treated by introducing a gene encoding an enzyme; that is, adenosine deaminase (ADA) or the like into cells of a patient with enzyme deficiency in adenosine deaminase (ADA) or the like.

In addition, in another embodiment, a method for preparing iPS cells is provided. The method for preparing iPS cells comprises an infection step for causing the virus vector according to the above Embodiment 2 to infect cells. The cells may be blood cells, inactivated (unstimulated) immunocytes, and are preferably naive T cells. Examples of the method for preparing iPS cells include methods for preparing iPS cells in a ground state.

### EXAMPLES

The present disclosure will be further specifically described with reference to the following examples, but the present disclosure is not limited by the examples.

### Cell Culture

Cell culture in the following examples was performed as follows. Mouse embryonic fibroblasts (MEF cells), Vero/SLAM cells, and BJ cells of a fibroblast cell line were cultured in a culture solution of Dulbecco's modified eagle's medium (DMEM) (NACALAI TESQUE, INC.) containing 10% fetal bovine serum (FBS) (HyClone). Vero/SLAM/F cells were cultured in a culture solution of DMEM containing 0.5 mg/mL G418 (NACALAI TESQUE, INC.) and 7.5% FBS.

BHK-T7/9 cells were cultured in a culture solution of a-modified minimum essential medium eagle (α-MEM) (Life Technologies Corporation) containing 600 μg/mL Hygromycin B (NACALAI TESQUE, INC.) and 10% FBS. Peripheral blood-derived blood cells of a healthy individual were cultured in a culture solution of RPMI1640 (NACALAI 1ESQUE, INC.) containing 10% FBS. Cord blood-derived or healthy individual-derived T cells were cultured in a culture solution of RPMI1640 containing 10% FBS, the same culture solution supplemented with 175 IU/mL human recombinant IL-2 (PeproTech, Inc.), or a KBM502 culture solution (Kohjin Bio Co., Ltd.). Cord blood-derived hematopoietic stem cells were cultured in StemSpan culture solution (Veritas Corporation) supplemented with human recombinant SCF, human recombinant Flt-3L and human recombinant TPO (all produced by PeproTech, Inc.).

All primed-type iPS cells were cultured on MEF cells in a culture solution for maintaining human ES cells. The composition of the culture solution for maintaining human ES cells is DMEM/F12 (NACALAI TESQUE, INC.) containing 20% KSR (Life Technologies), 2-mercaptoethanol (Sigma), 2 mM L-glutamine (NACALAI TESQUE, INC.), 1% non-essential amino acids (NACALAI TESQUE, INC.), and 4 ng/mL basic FGF (PeproTech, Inc.).

iPS cells in a ground state were cultured on MEF cells in a mixed culture solution. The composition of the mixed culture solution is DMEM/F12 containing 20% KSR, 2-mercaptoethanol, 2mM L-glutamine, 1% non-essential amino acid, 1 μM CHIR99021 (Militenyi Biotec K.K.), 1 μM PD0325901 (Militenyi Biotec K.K.), and 1,000 units/mL human recombinant LIF (NACALAI TESQUE, INC.).

### Example 1

### Construction of Non-Transmissible Gene-Modified Measles Virus Vector

Non-transmissible gene-modified measles virus vector was produced by co-culture of two plasmids (MV-HL-K-Pin1-EGFP and MV-NPM-OSM) containing genes encoding measles-virus-constituting functional proteins and four plasmids (pCITE-IC-N, pCITE-IC-PΔC, pCITEko-9301B-L, pCA7-IC-F) that help viral synthesis, with Vero/SLAM/F cells prepared as package cells by introducing a SLAM gene and the F gene of measles virus into BHK-T7/9 cells, as described below. As foreign genes, an OCT3/4 gene, an SOX2 gene, an L-MYC gene, a KLF4 gene and a PIN1 gene to be used for iPS cell preparation, and an EGFP gene as a reporter gene were inserted.

First, for insertion of N390I, N416D, N481Y, and E492G mutations into the H protein, and for insertion of P64S and E89K mutations into the M protein, wild-type H and M genes of a measles virus strain, MV-IC323-EGFP, were modified by genetic recombination.

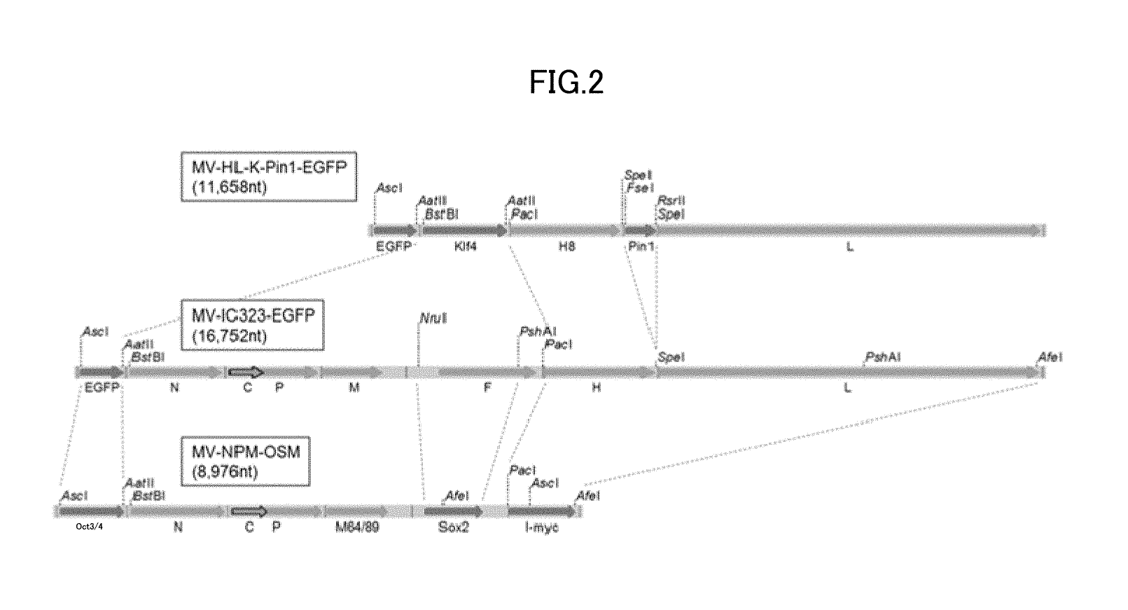

Subsequently, as shown in FIG. 2, the MV-NPM-OSM plasmid was prepared by recombining the EGFP gene of the above-mutated MV-IC323-EGFP with the OCT3/4 gene, the F gene of the same with the SOX2 gene, the H gene and the L gene of the same with the L-MYC gene. Meanwhile, the MV-HL-K-Pin1-EGFP plasmid was prepared by recombining the N gene, the P gene, the M gene and the F gene of the above mutated MV-IC323-EGFP with the KLF4 gene, and then inserting the PIN1 gene between the L gene and the H gene.

The MV-NPM-OSM plasmid and the MV-HL-K-Pin1-EGFP plasmid, and four plasmids (pCAG-T7-IC-F, pCITE-IC-N, pCITE-IC-PΔC, pCITE-ko-9301B-L) were used for transduction into BHK-T7/9 cells. Two days later, cells were recovered from dishes, and then seeded on Vero/SLAM/F cells. Two days later, giant cells were collected and a measles virus vector was recovered. The recovered measles virus vector was caused to infect fresh Vero/SLAM/F cells, and then after amplification, cells were recovered. Recovered cells were re-suspended in DMEM, followed by repeated freezing and thawing. Thus, the measles virus vector (MVV) was released from within cells into the culture solution. Next, a supernatant alone was recovered as a viral solution by centrifugation, dispensed, and then stored at −80° C. Titer was determined by adding the viral solution to Vero/SLAM cells, and then 2 days later, analyzing the proportion of GFP positive cells by a flow cytometric method.

### Example 2

### Evaluation of Non-Transmissible Gene-Modified Measles Virus Vector

MVV prepared in Example 1 was evaluated using activated T cells or BJ cells. Activated T cells were prepared by stimulating healthy individual-derived peripheral blood with Dynabeads (Registered Trademark) Human T-Activator CD3/CD28 (Life Technologies Corporation) in a KBM502 culture solution for 5 days. More specifically, 5×10⁴ cells of each cell type were seeded in a 12-well plate, and then a viral solution in an amount corresponding to the titer was added. Next, after transduction by a centrifugation method (room temperature, 1,200×g, 45 minutes), cells were washed once with PBS, and then culture solutions were exchanged. Two days later, GFP positive cells of activated T cells were analyzed for the expression of five genes (OCT3/4, SOX2, KLF4, L-MYC and PIN1) by a flow cytometric method. Moreover, BD Cytofix/Cytoperm (Trademark) Fixation/Permeabilization Kit (BD) was used to analyze the expression of proteins derived from foreign genes. Antibodies used herein were an anti-OCT3/4 antibody, an anti-SOX2 antibody, an anti-KLF4 antibody (all of these antibodies were produced by Santa cruz), and an anti-MYC antibody and an anti-PIN1 antibody (all of these antibodies were produced by R & D).

### Results

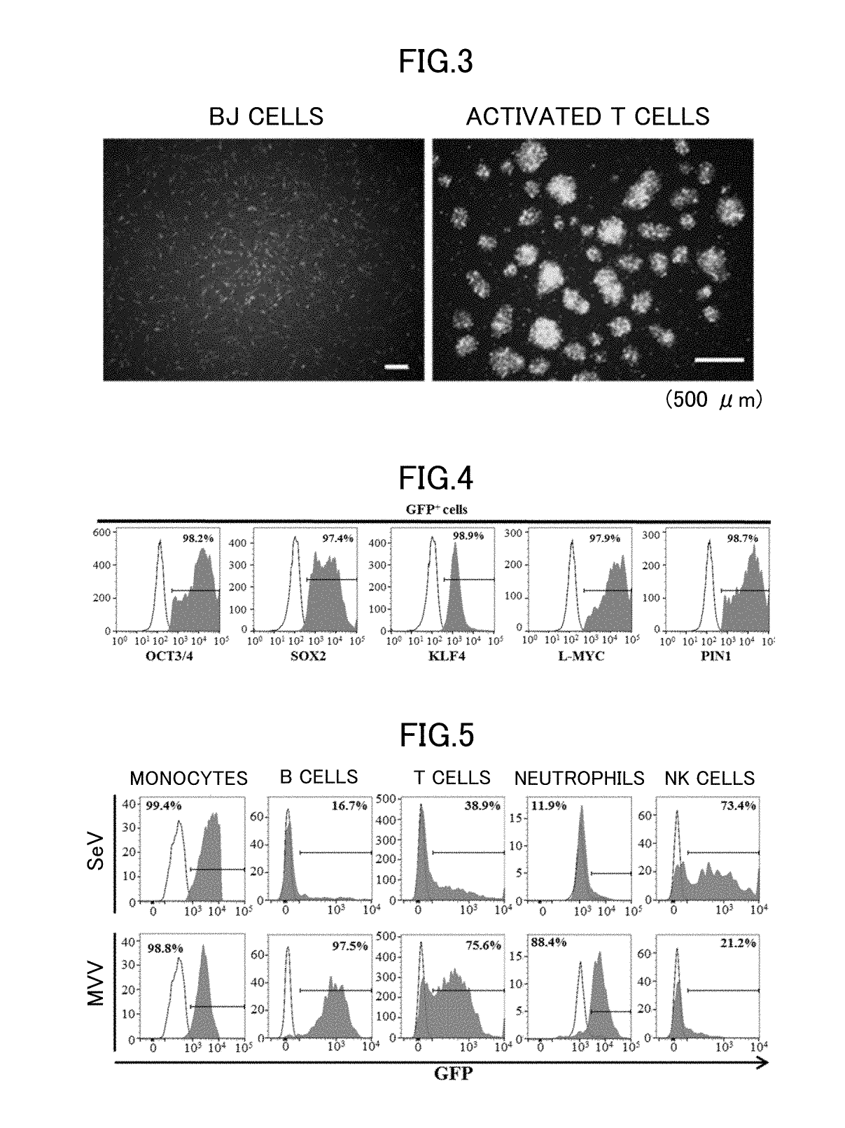

FIG. 3 shows fluorescent images of BJ cells and activated T cells. Fluorescence was confirmed for both cells. Hence, it could be confirmed that MVV conducts transduction into BJ cells and activated T cells.

FIG. 4 shows the results of analyzing GFP expression inactivated T cells. Transduction into activated T cells was performed using MVV. On day 3 after transduction, the expression of the above five genes in GFP-positive cells was analyzed. The simultaneous expression of all the five genes in addition to an EGFP gene could be confirmed from the results.

### Example 3

### Transduction by Non-Transmissible Gene-Modified Measles Virus Vector and Analysis Thereof

About 10 mL of peripheral blood or cord blood was collected from a healthy individual who had consented, and then mononuclear cells were separated using a lymphocyte separation solution (NACALAI TESQUE, INC.). At the same time, about 1 ml of peripheral blood or cord blood was lysed using a hemolytic agent prepared using NH₄Cl, KHCO₃ and EDTA (all produced by NACALAI TESQUE, INC.). Blood cells recovered by these methods were subjected to transduction (MOI=5) by centrifugation (room temperature, 1,200×g, 45 minutes) using MVV. After transduction, the resultant was washed with PBS(−) (prepared by dissolving PBS tablet (Takara Bio Inc.) with pure water, followed by autoclave sterilization), and then culture solutions were exchanged. Two days later, cells were recovered, and then the transduction rate of each blood cell lineage and the expression of the measles virus receptor in each cell lineage were analyzed by a flow cytometric method using GFP expression as an indicator.

As a control group for comparison, transduction was performed using a GFP gene-carrying Sendai virus vector (SeV) (PlasmEx (Registered Trademark)-AG; obtained from Medical and Biological Laboratories Co., Ltd.) by a method similar to that of MVV. Antibodies used for analysis of each blood cell lineage were monocyte lineage: an APC-Cy7-labeled anti-CD14 antibody (BioLegend, Inc.), a PE-labeled anti-CD11b antibody (BD), and a neutrophil-lineage: PerCP-Cy5.5-labeled anti-Cd15 antibody (BioLegend, Inc.), B cell lineage: an APC-Cy7-labeled anti-CD19 antibody (BioLegend, Inc.), T cell lineage: an APC-labeled anti-CD3 antibody (BioLegend, Inc.), an APC-Cy7-labeled anti-CD4 antibody (BioLegend, Inc.), a PE-Cy7-labeled anti-CD8 antibody (BioLegend, Inc.), an APC-labeled anti-CD45RA (BioLegend, Inc.), and a PE-labeled anti-CD197 antibody (Bio legend), and NK cells: a PE-Cy7-labeled anti-CD56 antibody (BioLegend, Inc.). Antibodies against viral receptors, which were used herein, are a PE-labeled anti-CD46 antibody (eBioscience), a PE-labeled anti-CD150 antibody (Bio legend), and a PE-labeled anti-Nectin4 antibody (R & D). In addition, monocytes were defined as CD14⁺ and CD11b⁺. B cells were defined as CD19⁺ and CD3⁻. T cells were defined as CD3⁺ and CD19⁻. Neutrophils were defined as CD15⁺. NK cells were defined as CD3⁻ and CD19⁻ and CD56⁺.

### Results

FIG. 5 shows the GFP positive rate of cells of each blood cell lineage derived from peripheral blood. Both vectors were confirmed to exert high transduction rates in monocytes, however, MVV was confirmed to have transduction efficiency higher than SeV in B cells, T cells and neutrophils.

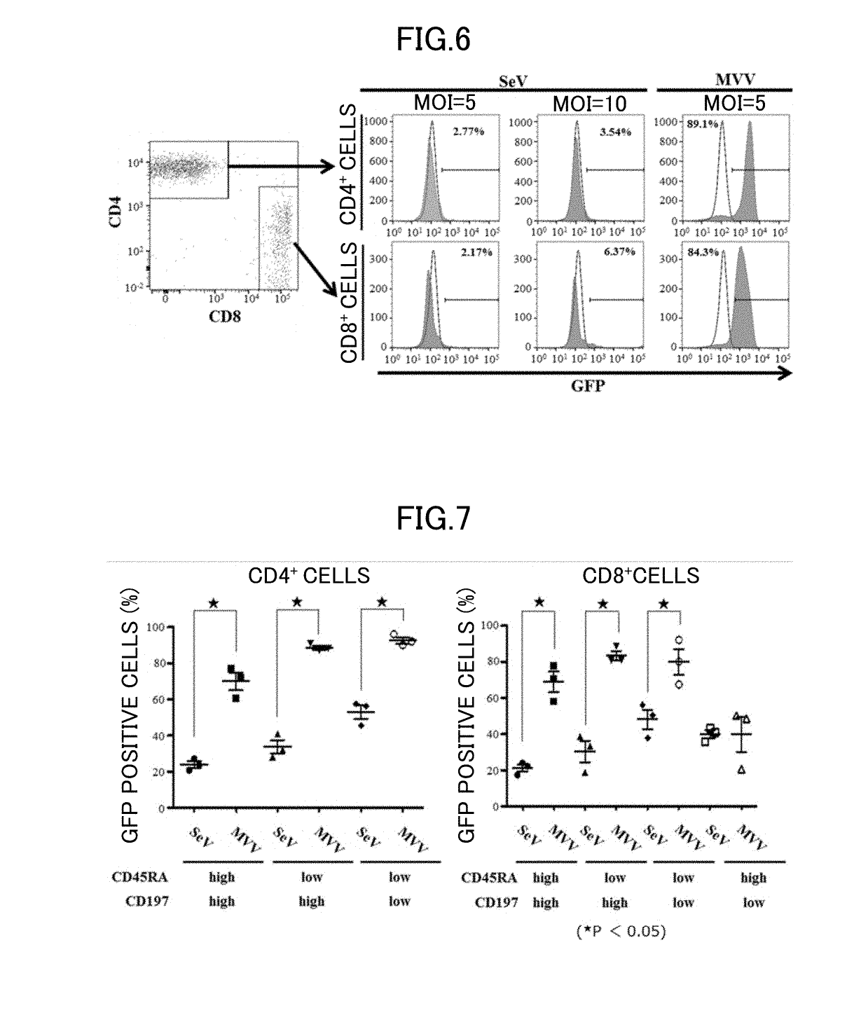

FIG. 6 shows GFP positive rates in inactivated T cells derived from cord blood. SeV was confirmed to perform no transduction even in the case of MOI=10, however, MVV exerted high transduction efficiency in the case of MOI=5.

T cells were fractionated into CD45RA and CCR7 (CD197), and each fraction was examined for transduction efficiency. As shown in FIG. 7, highly efficient transduction by MVV could be confirmed in fractions (CD45RA high and CD197 high) of naive T cells and stem cell-like memory T cells before exposed to antigens, and fractions (CD45RA low and CD197 high) of central memory T cells and fractions (CD45RA low and CD197 low) of effector memory T cells, which proliferate via re-exposure to antigens. On the other hand, from the results of CD8⁺ cells, no improvement in transduction efficiency by MVV was confirmed in effector T cell fractions (CD45RA high and CD low).

The above results indicate that MVV enables highly efficient transduction into naive T cells and stem cell-like memory T cells to which transduction has been difficult by conventional methods involving the use of Sendai virus vector. Therefore, it was strongly suggested that MVV according to this example can be an innovative therapeutic vector in a gene therapy using gene-modified T cells, and particularly a gene-modified T cell therapy. Furthermore, MVV could achieve highly efficient transduction into primary cultured B cells.

### Example 4

### Establishment of iPS Cells Using Non-Transmissible Gene-Modified Measles Virus Vector

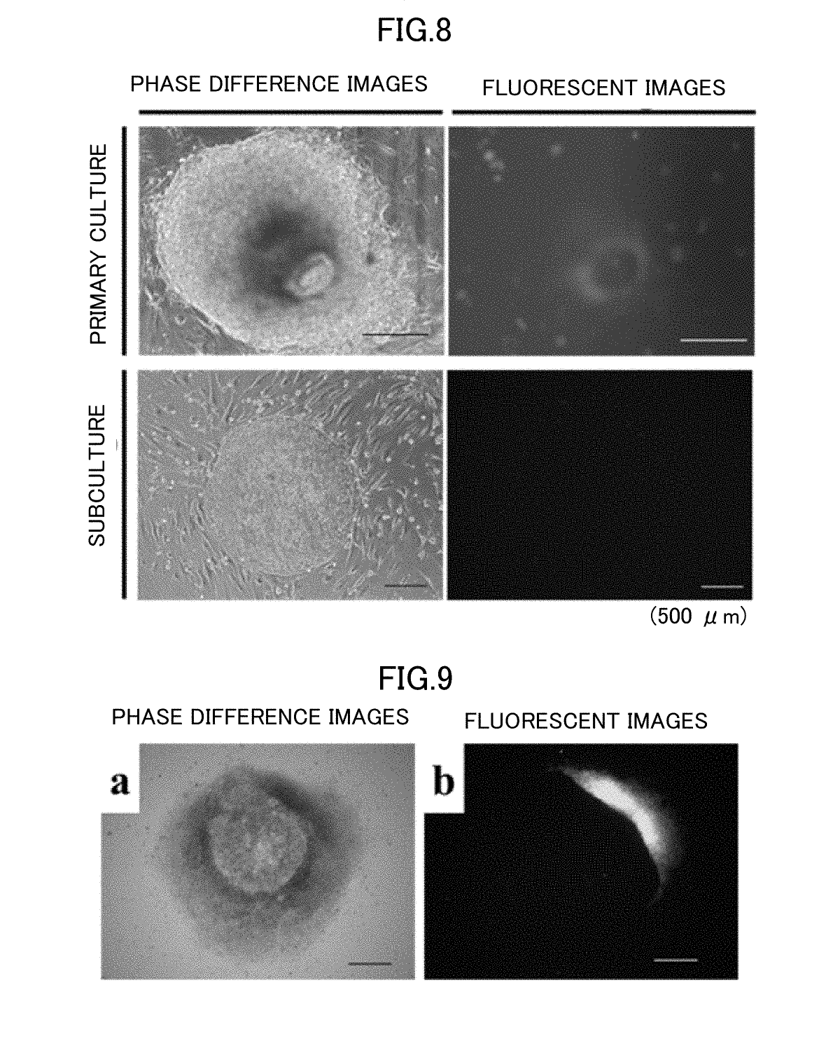

Transduction (MOI=5) was performed to BJ cells by centrifugation (room temperature, 1,200×g, 45 minutes) using MVV, the resultant was washed with PBS, and then culture solutions were exchanged. On day 7 after transduction, BJ cells were seeded on MEF cells, cultured for one day at 37° C. within a 5% CO₂ incubator, and then on the next day, the medium was exchanged with a culture solution for maintaining human ES cells. Cells were then cultured at 37° C. within a 5% CO₂ incubator while exchanging culture solutions every other day. On day 27 after transduction, human ES cell-like colonies were aliquoted (see FIG. 8).

Furthermore, peripheral blood-derived mononuclear cells of a healthy individual were stimulated in a KBM502 culture solution using Dynabeads (Registered Trademark) Human T-Activator CD3/CD28, and then cultured at 37° C. within a 5% CO₂ incubator. On day 5 after culture, cells were recovered, transduced with reprogramming factors by centrifugation (room temperature, 1,200×g, 45 minutes) using MVV, seeded on MEF cells on the next day and then cultured at 37° C. within a 5% CO₂ incubator. On day 20 after the start of culture, ES cell-like colonies (see FIG. 9) were recovered, and then seeded on fresh MEF cells.

The above iPS cells established from BJ cells were subjected to an immune antibody staining method and an RT-PCR method, thereby confirming the expression of an undifferentiation marker. The immune antibody staining method was performed by immobilizing human iPS cells using 4% paraformaldehyde-phosphate buffer (NACALAI TESQUE, INC.) at 4° C. for 30 minutes, treating the cells with 0.1% Triton X-100 (NACALAI TESQUE, INC.), and then blocking with 5% skim milk (NACALAI TESQUE, INC.). Staining was performed using as primary antibodies an anti-NANOG antibody (R & D), an anti-OCT3/4 antibody, an anti-SSEA-4 antibody, an anti-TRA-1-60 antibody and an anti-TRA-1-81 antibody (all antibodies produced by Santa cruz) at 4° C. overnight, and a secondary antibody (anti-goat InG (Life Technologies Corporation)) at room temperature for 30 minutes. Alkaline Phospatase Detection kit (Merck Millipore) was used for alkaline phosphatase staining.

With the RT-PCR method, RNA was extracted from the above iPS cells established from BJ cells using an RNeasy mini kit (Quiagen), and then a complementary strand DNA (cDNA) was synthesized using a SuperScript III First-Strand Synthesis System for RT-PCR (Life Technologies Corporation). Subsequently, an amplification reaction was performed using Takara ExTaq polymerase (Takara Bio Inc.), and then electrophoresis was performed using 1.5% agarose gel (NACALAI TESQUE, INC.). The nucleotide sequences of primers used in the RT-PCR method are SEQ ID NO: 3 and SEQ ID NO: 4 for Endo OCT3/4, SEQ ID NO: 5 and SEQ ID NO: 6 for Endo SOX2, SEQ ID NO: 7 and SEQ ID NO: 8 for Endo KLF4, SEQ ID NO: 9 and SEQ ID NO: 10 for EndoMYC, SEQ ID NO: 11 and SEQ ID NO: 12 for NANOG, SEQ ID NO: 13 and SEQ ID NO: 14 for TERT, SEQ ID NO: 15 and SEQ ID NO: 16 for DNMT3B, SEQ ID NO: 17 and SEQ ID NO: 18 for a MV-N protein, SEQ ID NO: 19 and SEQ ID NO: 20 for MV-L protein, and SEQ ID NO: 21 and SEQ ID NO: 22 for β-actin.

Induction of triploblastic differentiation by an in vitro embryoid formation method was examined. The above iPS cells established from BJ cells were dissociated from MEF using an ES cell dissociation solution (mixture of Collagenase IV (Life Technologies Corporation), 0.25% trypsin (Life Technologies Corporation) and KSR), re-suspended in a culture solution for maintaining human ES cells, seeded in a non-adhesive 6-well plate, and then cultured at 37° C. within a 5% CO₂ incubator. On the next day, nonadherent cells were recovered, re-suspended in a culture solution for maintaining human ES cells to which no basic FGF had been added, and then cultured at 37° C. within a 5% CO₂ incubator. On day 14 after the start of culture, cells were recovered, dissociated using a 0.25% trypsin/EDTA mixture (NACALAI TESQUE, INC.), seeded on dishes coated with a 0.1% gelatin solution (NACALAI TESQUE, INC.), and then cultured for 7 days at 37° C. within a 5% CO₂ incubator.

The above iPS cells established from BJ cells were confirmed to undergo triploblastic differentiation using an immune antibody staining method. Antibody used herein were an anti-alpha-fetoprotein antibody (R & D), an anti-vimentin antibody (Santa cruz) and an anti-nestin antibody (Santa cruz).

The above iPS cells established from BJ cells were subjected to a teratoma formation test. 1×10⁶ iPS cells were injected into the testis of each immunodeficient mouse (obtained from Central Institute for Experimental Animals). After 9 to 13 weeks, teratomas were extracted. Subsequently, teratoma tissues were fixed with 20% formalin (Wako Pure Chemical Industries, Ltd.), and then hematoxylin-eosin staining was performed. Moreover, the above iPS cells established from BJ cells were subjected to karyotype analysis (Chromocenter Inc.).

### Results

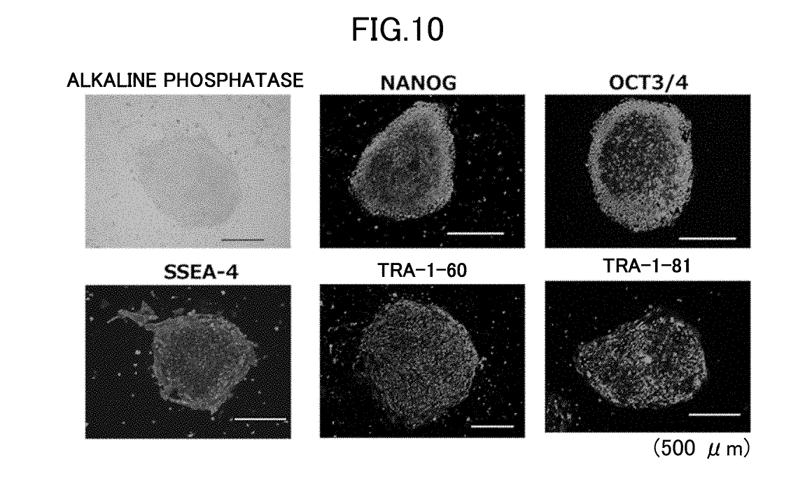

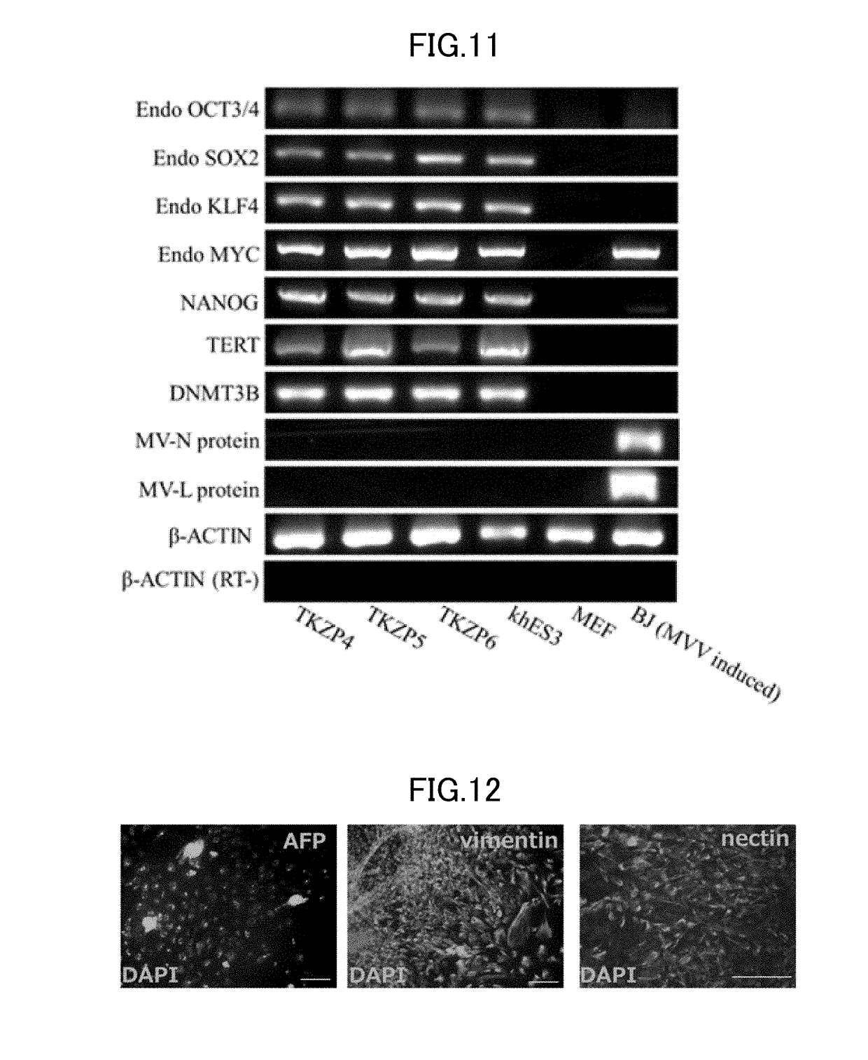

As shown in FIG. 10, the expression of alkaline phosphatase, NANOG, OCT3/4, SSEA-4, TRA-1-60 and TRA-1-81, which are expressed in undifferentiated cells, was confirmed in iPS cells. The results of the RT-PCR method are shown in FIG. 11. In each iPS cell sample, the expression of transduced OCT3/4, SOX2, KLF4 and L-MYC was confirmed. Furthermore, the expression of undifferentiation markers, NANOG, PERT and DNMT3B, was also confirmed. In addition, no expression of measles virus-derived N protein and L protein was observed. Moreover, the above iPS cells were confirmed to undergo triploblastic differentiation (see FIG. 12).

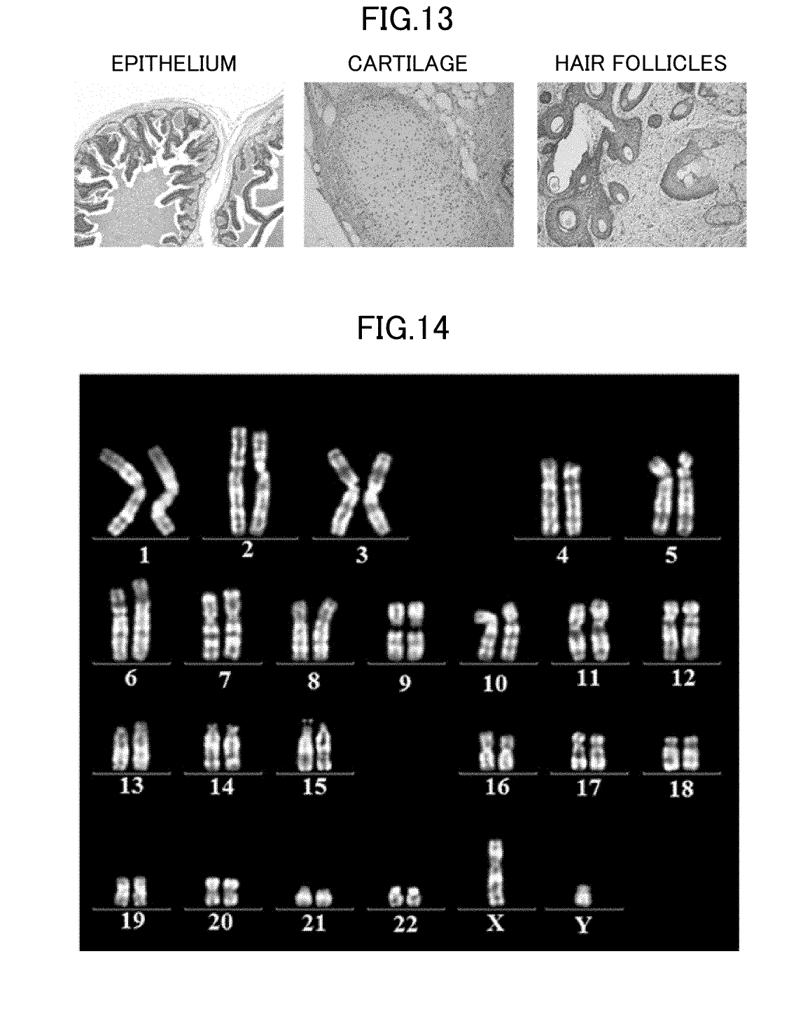

As a result of the teratoma formation test, as shown in FIG. 13, triploblastic differentiation ability was confirmed. This demonstrated the pluripotency of the above iPS cells established from BJ cells. As shown in FIG. 14, the iPS cells were found to have 46 chromosomes, and the karyotype was normal.

### Example 5

### Establishment, Induction of Differentiation, and Analysis of iPS Cells in a Ground State

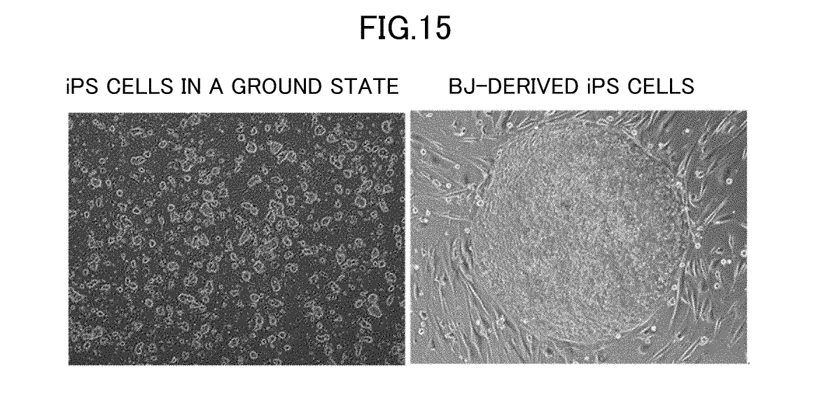

Cord blood-derived CD34 positive cells obtained from RIKEN were thawed, and then cultured using a culture solution prepared by adding SCF, TPO, and Flt3L to StemSpan at 37° C. within a 5% CO₂ incubator. On the next day, transduction was performed by centrifugation (room temperature, 1,200×g, 45 minutes) using MVV, and then cells were cultured in the above culture solution for 2 days. On day 3, the cells were seeded on MEF cells, and on day 4, the culture solution was exchanged with a culture solution for maintaining human ES cells. Subsequently, culture was continued at 37° C. within the 5% CO₂ incubator while exchanging culture solutions every other day. Colonies (see FIG. 15) appeared on day 14 after transduction were collected, and then dissociated into single cells with 0.25% trypsin/EDTA mixture. The cells were seeded on MEF cells and then cultured at 37° C. within the 5% CO₂ incubator.

### Results

As shown in FIG. 15, iPS cells in a ground state could be established. iPS cells in a ground state were dissociated into single cells at 4-day intervals using a 0.25% trypsin/EDTA mixture and then subcultured. As a result, colony morphology could be maintained for even 10 or more subcultures without using Y-27632. Furthermore, through exchange of the culture solution with a culture solution for maintaining human ES cells, so as to lower the concentration of cells to be seeded on MEF cells, appearance of human ES cell-like colonies could be confirmed.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

### INDUSTRIAL APPLICABILITY

The present disclosure is preferable for virus vectors and constructs for transduction to blood cells. Hence, the present disclosure is expected to be applied for immunotherapy, and particularly T cell therapy. Furthermore, the present disclosure is also preferable for preparation of pluripotent stem cells, and thus can be applied to, in addition to regenerative medicine using tissue cells obtained by induction of differentiation, disease analyses, drug discovery studies and the like using iPS cells established from cells derived from patients with intractable diseases. The present disclosure can further be applied to genome editing techniques targeting blood cells.

1. A virus vector, comprising a genome derived from a virus belonging to the family Paramyxoviridae in which both a gene encoding an H protein and a gene encoding an F protein are modified, wherein
the genome contains a foreign gene.

2. The virus vector according to claim 1, wherein
the genome is segmented into multiple segments, and
each genome segment comprises a leader sequence and a trailer sequence.

3. The virus vector according to claim 2, wherein
the genome is segmented into two segments.

4. The virus vector according to claim 1, wherein the modifications are
a deficiency in the gene encoding the H protein, or a mutation resulting from substitution, deletion, or addition of one or several amino acids of the H protein, and
a deficiency in the gene encoding the F protein, or a mutation resulting from substitution, deletion, or addition of one or several amino acids of the F protein.

5. The virus vector according to claim 1, wherein
a gene encoding an M protein in the genome comprises a mutation resulting from substitution, deletion, or addition of one or several amino acids of the M protein.

6. The virus vector according to claim 1, wherein
the virus belonging to the family Paramyxoviridae is measles virus.

7. The virus vector according to claim 1, wherein
the foreign gene includes OCT3/4, SOX2 and KLF4.

8. A cell, wherein
the foreign gene is introduced by the virus vector according to claim 1.

9. The cell according to claim 8, which is a blood cell including a hematopoietic stem cell.

10. The cell according to claim 9, wherein
the blood cell is a naive T cell, a stem cell-like memory T cell, or an undifferentiated B cell.

11. The cell according to claim 8, wherein
the foreign gene includes OCT3/4, SOX2 and KLF4, and
the cell is a pluripotent stem cell in a ground state.

12. A construct, containing
a nucleic acid that is a template for multiple genome segments derived from a virus belonging to the family Paramyxoviridae, in which a leader sequence is located at the 3′ end and a trailer sequence is located at the 5′ end, respectively, wherein
both the gene encoding the H protein and the gene encoding the F protein in the segmented genome are modified.


SEQUENCE LISTING (22 sequences)

SEQ ID NO: 1 - DNA, 8975 bp, Artificial sequence
  Segment 1 of modified measles virus genome
accaaacaaagttgggtaaggatagatcaatcaatgatcatattctagtacacttaggattcaagatcctattatcagggacaagagcaggattagggatatccgagggcgcgccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacattgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactgataagacgtcgagaggccgaggaccagaacaacatccgcctaccctccatcattgttataaaaaacttaggaaccaggtccacacagccgccagccaaccaaccatccactcccacgactggagttcgaacgagatggccacacttttgaggagcttagcattgttcaaaagaaacaaggacaaaccacccattacatcaggatccggtggagccatcagaggaatcaaacacattattatagtaccaattcctggagattcctcaattaccactcgatccagactactggaccggttggtcaggttaattggaaacccggatgtgagcgggcccaaactaacaggggcactaataggtatattatccttatttgtggagtctccaggtcaattgattcagaggatcaccgatgaccctgacgttagcatcaggctgttagaggttgttcagagtgaccagtcacaatctggccttaccttcgcatcaagaggtaccaacatggaggatgaggcggaccaatacttttcacatgatgatccaagcagtagtgatcaatccaggtccggatggttcgagaacaaggaaatctcagatattgaagtgcaagaccctgagggattcaacatgattctgggtaccattctagcccagatctgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctctttacgccgattcatggtggctctaatcctggatatcaagaggacacccgggaacaaacctaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaatctttaccagcaaatgggagaaactgcaccctacatggtaatcctagagaactcaattcagaacaagttcagtgcaggatcataccctctgctctggagctatgccatgggagtaggagtggaacttgaaaactccatgggaggtttgaactttggtcgatcttactttgatccagcatattttagattagggcaagagatggtgaggaggtcagctggaaaggtcagttccacattggcatccgaactcggtatcactgccgaggatgcaaggcttgtttcagagattgcaatgcatactactgaggacaggatcagtagagcggtcggacccagacaagcccaagtgtcatttctacacggtgatcaaagtgagaatgagctaccaggattggggggcaaggaagataggagggtcaaacagggtcggggagaagccagggagagctacagagaaaccgggtccagcagagcaagtgatgcgagagctgcccatcctccaaccagcatgcccctagacattgacactgcatcggagtcaggccaagatccgcaggacagtcgaaggtcagctgacgccctgctcaggctgcaagccatggcaggaatcttggaagaacaaggctcagacacggacacccctagggtatacaatgacagagatcttctagactaggtgcgagaggccgaggaccagaacaacatccgcctaccctccatcattgttataaaaaacttaggaaccaggtccacacagccgccagccaaccaaccatccactcccacgactggagccgatggcagaagagcaggcacgccatgtcaaaaacggactggaatgcatccgggctctcaaggccgagcccatcggctcactggccgtcgaggaagccatggcagcatggtcagaaatatcagacaacccaggacaggaccgagccacctgcaaggaagaggaggcaggcagttcgggtctcagcaaaccatgcctctcagcaattggatcaactgaaggcggtgcacctcgcatccgcggtcagggatctggagaaagcgatgacgacgctgaaactttgggaatcccctcaagaaatctccaggcatcaagcactgggttacagtgttatcatgtttatgatcacagcggtgaagcggttaagggaatccaagatgctgactctatcatggttcaatcaggccttgatggtgatagcaccctctcaggaggagacgatgaatctgaaaacagcgatgtggatattggcgaacctgataccgagggatatgctatcactgaccggggatctgctcccatctctatggggttcagggcttctgatgttgaaactgcagaaggaggggagatccacgagctcctgaaactccaatccagaggcaacaactttccgaagcttgggaaaactctcaatgttcctccgcccccgaaccccagtagggccagcacttccgagacacccattaaaaagggcacagacgcgagattggcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctccgatgtccaagacatcaaaacagccttggccaaaatacacgaggataatcagaagataatctccaagctagaatcattgctgttattgaagggagaagttgagtcaattaagaagcagatcaacaggcaaaatatcagcatatccaccctggaaggacacctctcaagcatcatgattgccattcctggacttgggaaggatcccaacgaccccactgcagatgtcgaactcaatcccgacctgaaacccatcataggcagagattcaggccgagcactggccgaagttctcaagaagcccgttgccagccgacaactccagggaatgactaatggacggaccagttccagaggacagctgctgaaggaatttcaactaaagccgatcgggaaaaaggtgagctcagccgtcgggtttgttcctgacaccggccctgcatcacgcagtgtaatccgctccattataaaatccagccggctagaggaggatcggaagcgttacctgatgactctccttgatgatatcaaaggagccaacgatcttgccaagttccaccagatgctgatgaagataataatgaagtagctacagctcaacttacctgccaaccccatgccagtcgacctaattagtacaacctaaatccattataaaaaacttaggagcaaagtgattgcctcctaagttccacaatgacagagatctacgatttcgacaagtcggcatgggacatcaaagggtcgatcgctccgatacaacctaccacctacagtgatggcaggctggtgccccaggtcagagtcatagatcctggtctaggtgataggaaggatgaatgctttatgtacatgtttctgctgggggttgttgaggacagcgatcccctagggcctccaatcgggcgagcattcgggtccctgcccttaggtgttggtagatccacagcaaaacccgaggaactcctcaaagaggccactgagcttgacatagttgttagacgtacagcagggctcaatgaaaaactggtgttctacaacaacaccccactaaccctcctcacaccttggagaaaggtcctaacaacagggagtgtcttcaatgcaaaccaagtgtgcaatgcggttaatctaataccgctggacaccccgcagaggttccgtgttgtttatatgagcatcacccgtctttcggataacgggtattacaccgttcccagaagaatgctggaattcagatcggtcaatgcagtggccttcaacctgctagtgacccttaggattgacaaggcgattggccctgggaagatcatcgacaatgcagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccactgatggatatcaatgaagaccttaatcggttactctggaggagcagatgcaagatagtaagaatccaggcagttttgcagccatcagttcctcaagaattccgcatttacgacgacgtgatcataaatgatgaccaaggactattcaaagttctgtagaccgcagtgcccagcaatacccgaaaacgacccccctcataatgacagccagaaggcccggacaaaaaagccccctccagaagactccacggaccaagcgagaggccagccagcagccgacagcaagtgtggacaccaggcggcccaagcacagaacagccccgacacaaggccaccaccagccatcccaatctgcgtcctcctcgtgggacccccgaggaccaaccccgaaggtcgctccgaacacagaccaccaaccgcatccccacagctcccgggaaaggaacccccagcaactggaaggcccctccccccctcccccaacgcaagaaccccacaaccgaaccgcacaagcgaccgaggtgacccaaccgcaggcatccgactccttagacagatcctctccccccggcatactaaacaaaacttagggccaaggaacacacacactcgacagaacccagaccccggcccgcggcaccgcgcccccaccccccgaaaaccagagggagcccccaaccaaacccgccggcccccccggtgcccacaggtaggcacaccaacccccgaccagacccagcacccagccaccgacaatccaagacggggggccccccccaaaaaaaggcccccaggggccgacagccagcatcgatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgataacggtcctcccatatcattggagaggttggacgtagggacaaatctggggaatgcaattgctaagttggaggatgccaaggaattgttggagtcatcggaccagatattgaggagtatgaaaggtttatcgagcactagcatagtttacatcctgattgcagtgtgtcttggagggttgatagggatccccgctttaatatgttgctgcagggggcgctgtaacaaaaagggagaacaagttggtatgtcaagaccaggcctaaagcctgatcttacagggacatcaaaatcctatgtaaggtcgctctgatcctctacaactcttgaaacacagatttcccacaagtctcctcttcgtcatcaagcaaccaccgcatccagcatcaagcccacctgaaattgtctccggcttccctctggccgaacgatatcggtagttaattaaaacttagggtgcaagatcatccacaatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtcgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctactaatagcgctctgattaaggattaattggttgaactccggaaccctaatcctgccctaggtagttaggcattatttgcaatatattaaagaaaactttgaaaatacgaagtttctattcccagctttgtctggt

SEQ ID NO: 2 - DNA, 11658 bp, Artificial sequence
  Segment 2 of modified measles virus genome
accaaacaaagttgggtaaggatagatcaatcaatgatcatattctagtacacttaggattcaagatcctattatcagggacaagagcaggattagggatatccgagggcgcgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagacgtcgagaggccgaggaccagaacaacatccgcctaccctccatcattgttataaaaaacttaggaaccaggtccacacagccgccagccaaccaaccatccactcccacgactggagttcgaagcagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaataggacgtcgagaggccgaggaccagaacaacatccgcctacgatatcggtagttaattaaaacttagggtgcaagatcatccacaatgtcaccacaacgagaccgaataaatgccttctacaaagacaacccacatcctaagggaagtaggatagttattaacagagaacatcttatgattgatagaccttatgttttgctggctgttctattcgtcatgtttctgagcttgatcgggttgctagccattgcaggcattagactccatcgtgcagccatctacaccgcagagatccataagagcctcagcaccaatctagatgtaactaactcgatcgagcatcaggtcaaggacgtgctgacaccactcttcaagatcattggtgatgaagtgggcctgaggacacctcagagattcactgacctagtgaaattcatctctgacaaaattaaattccttaatccggatagggagtacgacttcagagatctcacttggtgtatcaacccgccagagagaatcaaattggattatgatcaatactgtgcagatgtggctgctgaagaactcatgaatgcattggtgaactcaactctactggaggccagggcaaccaatcagttcctagctgtctcaaagggaaactgctcagggcccactacaatcagaggtcaattctcaaacatgtcgctgtccctgttggacttgtatttaagtcgaggttacaatgtgtcatctatagtcactatgacatcccagggaatgtacgggggaacttacctagtgggaaagcctaatctgagcagtaaagggtcagagttgtcacaactgagcatgcaccgagtgtttgaagtaggggttatcagaaatccgggtttgggggctccggtgttccatatgacaaactattttgagcaaccagtcagtaatgatttcagcaactgcatggtggctttgggggagcttaaattcgcagccctctgtcacagggaagattctatcacaattccctatcaggggtcagggaaaggtgtcagcttccagctcgtcaagctaggtgtctggaaatccccaaccgacatgcgatcctgggtccccctatcaacggatgatccagtgatagataggctttatctctcatctcacagaggtgttatcgctgacaatcaagcaaaatgggctgtcccgacaacacggacagatgacaagttgcgaatggagacatgcttccagcaggcgtgtaagggtaaaatacaagcactctgcgagaatcccgagtgggcaccattgaaggataacaggattccttcatacggggtcttgtctgttgacctgagtctgacagttgagcttaaaatcaaaattgcttcaggattcgggccattgatcacacacggttcagggatggacctatacaaaaccaaccacaacaatgtgtattggctgactatcccgccaatgaagaacctagccttaggtgtaatcaacacattggagtggataccgagattcaaggttagtccctatctcttcactgttccaatcaaggaagcaggcggcgactgccatgccccaacatacctacctgcggaggtggatggtgatgtcaaactcagttccaatctggtaattctacctggtcaggatctccaatatgttttggcaacctacgatacttccagggttgaacatgctgtggtttattatgtttacagcccaagccgctcattttcttacttttatccttttaggttgcctataaagggggtcccaatcgaattacaagtggaatgcttcacatgggacaaaaaactctggtgccgtcacttctgtgtgcttgcggactcagaatctggtggacatatcactcactctgggatggtgggcatgggagtcagctgcacagtcactcgggaagatggaaccaatcgcagatagggctgccagtgaaccgatcacatgatgtcacccagacatcaggcatacccactagtgtgaaatagacatcagaattaagaaaaacgtagggtccaagtggtttcccgttggccggccatggcggacgaggagaagctgccgcccggctgggagaagcgcatgagccgcagctcaggccgagtgtactacttcaaccacatcactaacgccagccagtgggagcggcccagcggcaacagcagcagtggtggcaaaaacgggcagggggagcctgccagggtccgctgctcgcacctgctggtgaagcacagccagtcacggcggccctcgtcctggcggcaggagaagatcacccggaccaaggaggaggccctggagctgatcaacggctacatccagaagatcaagtcgggagaggaggactttgagtctctggcctcacagttcagcgactgcagctcagccaaggccaggggagacctgggtgccttcagcagaggtcagatgcagaagccatttgaagacgcctcgtttgcgctgcggacgggggagatgagcgggcccgtgttcacggattccggcatccacatcatcctccgcactgagtgacggaccgacccactagtgtgaaatagacatcagaattaagaaaaacgtagggtccaagtggtttcccgttatggactcgctatctgtcaaccagatcttataccctgaagttcacctagatagcccgatagttaccaataagatagtagctatcctggagtatgctcgagtccctcacgcttacagcctggaggaccctacactgtgtcagaacatcaagcaccgcctaaaaaacggattctccaaccaaatgattataaacaatgtggaagttgggaatgtcatcaagtccaagcttaggagttatccggcccactctcatattccatatccaaattgtaatcaggatttatttaacatagaagacaaagagtcaacaaggaagatccgtgagctcctaaaaaagggaaattcgctgtactccaaagtcagtgataaggttttccaatgcctgagggacactaactcacggcttggcctaggctccgaattgagggaggacatcaaggagaaaattattaacttgggagtttacatgcacagctcccaatggtttgagccctttctgttttggtttacagtcaagactgagatgaggtcagtgattaaatcacaaacccatacttgccataggaggagacacacacctgtattcttcactggtagttcagttgagctgttaatctctcgtgaccttgttgctataatcagtaaggaatctcaacatgtatattacctgacgtttgaactggttttgatgtattgtgatgtcatagaggggaggttaatgacagagaccgctatgaccattgatgctaggtatgcagaacttctaggaagagtcagatacatgtggaaactgatagatggtttcttccctgcactcgggaatccaacttatcaaattgtagccatgctggagccactttcacttgcttacctgcaactgagggatataacagtagaactcagaggtgctttccttaaccactgctttactgaaatacatgatgttcttgaccaaaacgggttttctgatgaaggtacttatcatgagttaattgaagccctagattacattttcataactgatgacatacatctgacaggggagattttctcatttttcagaagtttcggccaccccagacttgaagcagtaacggctgctgaaaatgtcaggaaatacatgaatcagcctaaagtcattgtgtatgagactctgatgaaaggtcatgccatattttgtggaatcataatcaacggctatcgtgacaggcacggaggcagttggccacccctgaccctccccctgcatgctgcagacacaatccggaatgctcaagcttcaggtgaagggttaacacatgagcagtgcgttgataactggaaatcatttgctggagtgagatttggctgttttatgcctcttagcctggacagtgatctgacaatgtacctaaaggacaaggcacttgctgctctccaaagggaatgggattcagtttacccgaaagagttcctgcgttacgatcctcccaagggaaccgggtcacggaggcttgtagatgttttccttaatgattcgagctttgacccatatgatatgataatgtatgtcgtaagtggagcctacctccatgaccctgagttcaacctgtcttacagcctgaaagaaaaggagatcaaggaaacaggtagacttttcgctaaaatgacttacaaaatgagggcatgccaagtgatcgctgaaaatctaatctcaaacgggattggcaagtattttaaggacaatgggatggccaaggatgagcacgatttgactaaggcactccacactctggctgtctcaggagtccccaaagatctcaaagaaagtcacaggggggggccagtcttaaaaacctactcccgaagcccagtccacacaagtaccaggaacgttaaagcagaaaaagggtttgtaggattccctcatgtaattcggcagaatcaagacactgatcatccggagaatatagaaacctacgagacagtcagcgcatttatcacgactgatctcaagaagtactgccttaattggagatatgagaccatcagcttatttgcacagaggctaaatgagatttacggattaccctcattttttcagtggctgcataagaggcttgaaacctctgtcctctatgtaagtgaccctcattgcccccccgaccttgacgcccatgtcccgttatgcaaagtccccaatgaccaaatcttcatcaagtaccctatgggaggtatagaagggtattgtcagaagctgtggaccatcagcaccattccctacttatacctggctgcttatgagagccgggtaaggattgcttcgttagtgcaaggggacaatcagaccatagccgtaacaaaaagggtacccagcacatggcattacaaccttaagaaacgggaagctgctagagtaactagagattactttgtaattcttaggcaaaggctacatgacattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcggcatgcagtaatattgctacaacaatggccaaaagcattgagagaggttatgaccgttatcttgcatattccctgaacgtcctaaaagtgatacagcaaattttgatctcccttggcttcacaatcaattcaaccatgacccgggatgtagtcatacccctcctcacaaacaacgatctcttaataaggatggcactgttgcccgctcctattgggggtatgaattatctgaatatgagcaggctgtttgtcagaaacatcggtgatccagtaacatcatcaattgctgatctcaagagaatgattctcgcatcactaatgcctgaagagaccctccatcaagtaatgacacaacaaccgggggactcttcattcctagactgggctagcgacccttactcagcaaatcttgtatgcgtccagagcatcactagactcctcaagaacataactgcaaggtttgtcctgatccacagtccaaacccaatgttaaaagggttattccatgatgacagcaaagaagaggacgagagactggcggcattcctcatggacaggcatattatagtacctagggcagctcatgaaatcctggatcatagtgtcacaggggcaagagagtctattgcaggcatgctagataccacaaaaggcctgattcgagccagcatgaggaagggggggttaacctctcgagtgataaccagattgtccaattatgactatgaacaatttagagcagggatggtgctattgacaggaagaaagagaaatgtcctcattgacaaagagtcatgttcagtgcagctggctagagccctaagaagccatatgtgggcaagactagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagctcgccttcgtaagagccccaagtagatccttgcgatctgccgttagaatagcaacagtgtactcatgggcttacggtgatgatgatagctcttggaacgaagcctggttgttggcaaggcaaagggccaatgtgagcctggaggagctaagggtgatcactcccatctcgacttcgactaatttagcgcataggttgagggatcgtagcactcaagtgaaatactcaggtacatcccttgtccgagtggcaaggtataccacaatctccaacgacaatctctcatttgtcatatcagataagaaggttgatactaactttatataccaacaaggaatgcttctagggttgggtgttttagaaacattgtttcgactcgagaaagatactggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattctagctaagtccacagcactatctatgattgacctggtaacaaaatttgagaaggaccatatgaatgaaatttcagctctcataggggatgacgatatcaatagtttcataactgagtttctgcttatagagccaagattattcaccatctacttgggccagtgtgcagccatcaattgggcatttgatgtacattatcatagaccatcagggaaatatcagatgggtgagctgttgtcttcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttcttttgtgtgaaagcgatgaggatgtagtaccggacagattcgacaacatccaggcaaaacacttgtgtgttctggcagatttgtactgtcaaccagggacctgcccaccgattcgaggtctaaggccggtagagaaatgtgcagttctaaccgatcatatcaaggcagaggctaggttatctccagcaggatcttcgtggaacataaatccaattattgtagaccattactcatgctctctgacttatctccgtcgaggatctatcaaacagataagattgagagttgatccaggattcatttttgacgccctcgctgaggtaaatgtcagtcagccaaaggtcggcagcaacaacatctcaaatatgagcatcaaggatttcagacctccacacgatgatgttgcaaaattgctcaaagatatcaacacaagcaagcacaatcttcccatttcagggggtagtctcgccaattatgaaatccatgctttccgcagaatcgggttaaactcatctgcttgctacaaagctgttgagatatcaacattaattaggagatgccttgagccaggggaagacggcttgttcttgggtgaggggtcgggttctatgttgatcacttataaggagatactaaaactaaacaagtgcttctataatagtggggtttccgccaattctagatctggtcaaagggaattagcaccctatccctccgaagttggccttgtcgaacacagaatgggagtaggtaatattgtcaaggtgctctttaacgggaggcccgaagtcacgtgggtaggcagtatagattgcttcaatttcatagtcagtaatatccctacctctagtgtggggtttatccattcagatatagagaccttacctaacaaagatactatagagaagctagaggaattggcagccatcttatcgatggctctactccttggcaaaataggatcaatactggtgattaagcttatgcctttcagcggggattttgttcagggatttataagctatgtagggtctcattatagagaagtgaaccttgtctaccctaggtacagcaacttcatatctactgaatcttatttagttatgacagatctcaaagctaaccggctaatgaatcctgaaaagatcaagcagcagataattgaatcatctgtacggacttcacctggacttataggtcacatcctatccattaagcaactaagctgcatacaagcaattgtgggaggcgcggttagtagaggtgatatcaaccctattctgaaaaaacttacacctatagagcaggtgctgatcagttgcgggttggcaattaacggacctaaactgtgcaaagaattaatccaccatgatgttgcctcagggcaagatggattgcttaactctatactcatcctctacagggagttggcaagattcaaagacaaccaaagaagtcaacaagggatgttccacgcttaccccgtattggtaagtagtaggcaacgagaacttgtatctaggatcactcgcaaattttgggggcatattcttctttactccgggaacagaaagttgataaatcggtttatccagaatctcaagtccggttatctagtactagacttacaccagaatatcttcgttaagaatctatccaagtcagagaaacagattattatgacggggggtttaaaacgtgagtgggtttttaaggtaacagtcaaggagaccaaagaatggtacaagttagtcggatacagcgctctgattaaggattaattggttgaactccggaaccctaatcctgccctaggtagttaggcattatttgcaatatattaaagaaaactttgaaaatacgaagtttctattcccagctttgtctggt

SEQ ID NO: 3 - DNA, 24 bp, Artificial sequence
  PCR primer for Endo OCT3/4, S1165
gacagggggaggggaggagctagg

SEQ ID NO: 4 - DNA, 26 bp, Artificial sequence
  PCR primer for Endo OCT3/4, AS1283
cttccctccaaccagttgccccaaac

SEQ ID NO: 5 - DNA, 26 bp, Artificial sequence
  PCR primer for Endo SOX2, S1430
gggaaatgggaggggtgcaaaagagg

SEQ ID NO: 6 - DNA, 26 bp, Artificial sequence
  PCR primer for Endo SOX2, AS1555
ttgcgtgagtgtggatgggattggtg

SEQ ID NO: 7 - DNA, 25 bp, Artificial sequence
  PCR primer for Endo KLF4, S1128
acgatcgtggccccggaaaaggacc

SEQ ID NO: 8 - DNA, 28 bp, Artificial sequence
  PCR primer for Endo KLF4, AS1826
tgattgtagtgctttctggctgggctcc

SEQ ID NO: 9 - DNA, 26 bp, Artificial sequence
  PCR primer for Endo MYC, S253
gcgtcctgggaagggagatccggagc

SEQ ID NO: 10 - DNA, 26 bp, Artificial sequence
  PCR primer for Endo MYC, AS555
ttgaggggcatcgtcgcgggaggctg

SEQ ID NO: 11 - DNA, 25 bp, Artificial sequence
  PCR primer for NANOG, 968S
cagccccgattcttccaccagtccc

SEQ ID NO: 12 - DNA, 25 bp, Artificial sequence
  PCR primer for NANOG, 1334AS
cggaagattcccagtcgggttcacc

SEQ ID NO: 13 - DNA, 26 bp, Artificial sequence
  PCR primer for TERT, S3234
cctgctcaagctgactcgacaccgtg

SEQ ID NO: 14 - DNA, 25 bp, Artificial sequence
  PCR primer for TERT, AS3713
ggaaaagctggccctggggtggagc

SEQ ID NO: 15 - DNA, 25 bp, Artificial sequence
  PCR primer for DNMT3B, S2502
tgctgctcacagggcccgatacttc

SEQ ID NO: 16 - DNA, 28 bp, Artificial sequence
  PCR primer for DNMT3B, S2716
tcctttcgagctcagtgcaccacaaaac

SEQ ID NO: 17 - DNA, 18 bp, Artificial sequence
  PCR primer for MV-N protein, FW
gaactcggtatcactgcc

SEQ ID NO: 18 - DNA, 18 bp, Artificial sequence
  PCR primer for MV-N protein, RV
tcctggtagctcattctc

SEQ ID NO: 19 - DNA, 20 bp, Artificial sequence
  PCR primer for MV-L protein, FW
cacggtattacatcttcacg

SEQ ID NO: 20 - DNA, 20 bp, Artificial sequence
  PCR primer for MV-L protein, RV
gcatctctgtcaattaaagg

SEQ ID NO: 21 - DNA, 21 bp, Artificial sequence
  PCR primer for beta-actin, S756
gcaagagatggccacggctgc

SEQ ID NO: 22 - DNA, 22 bp, Artificial sequence
  PCR primer for beta-actin, AS1033
tctccttctgcatcctgtcggc